(19) United States
(12) Patent Application Publication
(10) Pub. No.: US 2010/0254902 A1
(43) Pub. Date: Oct. 7, 2010

(54) STABILIZATION OF RADIOPHARMACEUTICALS

(76) Inventor: Jan Van Den Bos, Eindhoven (NL)

Correspondence Address: GE HEALTHCARE BIO-SCIENCES CORP.;PATENT DEPARTMENT, 101 CARNEGIE CENTER, PRINCETON, NJ, 08540, US

(21) Appl. No.: 12/741,099

(22) PCT Filed: Nov. 4, 2008
(86) PCT No.: PCT/EP08/64953

§ 371 Date: May 3, 2010

(51) Int. Cl. A61K 51/04; A61K 51/00; A61P 43/00

(52) U.S. Cl. 424/1.73; 424/1.11; 424/1.89

(57) ABSTRACT

The invention relates to stabilised radiopharmaceutical compositions which comprise: (i) an ¹⁸F-labelled compound; (ii) an effective stabilizing amount of gentisic acid or a salt thereof with a biocompatible cation; (iii) an aqueous biocompatible carrier medium; wherein the radioactive concentration of the ¹⁸F in the carrier medium is in the range 10 to 100,000 MBq/ml and the pH of the composition is in the range 4.0 to 9.5. The invention further comprises methods for preparing such radiopharmaceutical compositions and a new use of gentisic acid or a salt thereof.

The present invention relates to stabilised ¹⁸F-labelled radiopharmaceutical compositions, to methods for their preparation, and to a new use of gentisic acid or a salt thereof.

¹⁸F has a half-life of 109.7 minutes which means that ¹⁸F-radiopharmaceuticals are produced as close as possible to the site of clinical use and in relatively large batches to allow for decay during delivery to the patient. The practice of terminal sterilization of radiopharmaceuticals using an autoclave cycle further leads to instability of ¹⁸F-radiopharmaceuticals. The generally accepted mechanism of defluoridation of a ¹⁸F-labelled radiopharmaceutical in vitro is radiolysis of the ¹⁸F-imaging agent in aqueous solution. In aqueous media, radioactive decay causes the formation of highly-reactive oxygen species that react with organic molecules. The reactive species arise from degradation of the water solvent, and are free radicals such as hydroxyl or superoxide free radicals.

Gentisic acid has previously been disclosed as a stabiliser for use in lyophilised kits for the preparation of ^{99m}Tc radiopharmaceuticals, for example in U.S. Pat. No. 4,497,744.

WO 02/04030 describes stable radiopharmaceutical compositions, comprising a radiopharmaceutical (where the radioisotope is selected from ^{99m}Tc, ¹³¹I, ¹²⁵I, ^{117m}Sn, ¹¹¹In, ⁹⁷Ru, ²⁰³Pb, ⁶⁷Ga, ⁶⁸Ga, ⁸⁹Zr, ⁹⁰Y, ¹⁷⁷Lu, ¹⁴⁹Pm, ¹⁵³Sm, ¹⁶⁶Ho, ³²P, ²¹¹At, ⁴⁷Sc, ¹⁰⁹Pd, ¹⁰⁵Rh, ¹⁸⁶Re, ¹⁸⁸Re, ⁶⁰Cu, ⁶²Cu, ⁶⁴Cu, and ⁶⁷Cu) and an effective stabilizing amount of a substituted aromatic compound.

Use of gentisic acid and salts thereof for stabilization of radioiodinated radiopharmaceuticals has been described in WO2007/007021.

Use of radical traps, such as gentisic acid, to improve yields in radiofluoridation of iodonium salts has been disclosed in WO2005/061415.

Stabilized formulations of ¹⁸F-labelled radiopharmaceuticals have been described in the art, particularly stabilized formulations of 2-[¹⁸F]Fluoro-2-deoxy-D-glucose ([¹⁸F]FDG) addressing the problem of radiolysis. For example, WO2004/043497 describes stabilization of an [¹⁸F]FDG radiopharmaceutical using ethyl alcohol, and WO 03/090789 describes a method for improving one or more physical/chemical characteristics, such as reduced radiolysis and the ability to autoclave an [¹⁸F]FDG solution by addition of buffer.

Use of ¹⁸F-labelled radiopharmaceuticals in the clinic is increasing rapidly with the uptake of the in vivo imaging method positron emission tomography (PET), in particular use of [¹⁸F]FDG as a radioimaging agent for clinical research or for diagnostic purposes has increased significantly in recent years. Accordingly, to meet this high demand, there is a need to manufacture ¹⁸F-labelled radiopharmaceuticals such as [¹⁸F]FDG in larger batch sizes which in turn makes it more difficult to routinely prepare batches which meet with the radiochemical purity (RCP) standards required by Regulatory authorities (see for example, European Pharmacopoeia 01/2005:1325). Therefore, there still exists a need for further methods for stabilizing ¹⁸F-labelled radiopharmaceuticals, for example, [¹⁸F]FDG.

In a first aspect, the present invention provides a stabilised radiopharmaceutical composition which comprises:
(i) an ¹⁸F-labelled compound;
(ii) an effective stabilizing amount of gentisic acid or a salt thereof with a biocompatible cation;
(iii) an aqueous biocompatible carrier medium;
   wherein the radioactive concentration of the ¹⁸F in the carrier medium is in the range 10 to 100,000 MBq/ml and the pH of the composition is in the range 4.0 to 9.5.

The term “¹⁸F-labelled compound” means an ¹⁸F-labelled compound which is suitable for detection by PET imaging within a mammalian subject, suitably a human. The ¹⁸F-labelled compound is preferably a non-peptide. By the term “non-peptide” is meant a compound which does not comprise any peptide bonds, i.e. an amide bond between two amino acid residues.

Suitable ¹⁸F-labelled compounds include [¹⁸F]FDG, [¹⁸F]-fluoro-DOPA, [¹⁸F]-fluoroestradiol, 3′-[¹⁸F]-fluorothymidine, 5-[¹⁸F]fluorouracil, [¹⁸F]fluorodopamine, [¹⁸F]fluoronorepinephrine, 2β-carbomethoxy-3β-(4-iodophenyl)nortropane ([¹⁸F]CFT), N—[¹⁸F]-fluoropropyl-2β-carbomethoxy-3β-(4-iodophenyl)nortropane ([¹⁸F]FP-CFT), 2-(1-(6-((2-[¹⁸F]-fluoroethyl)(methyl)amino)naphthalen-2-yl)ethylidene)malonitrile ([¹⁸F]FDDNP), 2-(3-[¹⁸F]-fluoro-4-methylamino-phenyl)-benzothiazol-6-ol, 2-(2-[¹⁸F]-fluoro-4-methylamino-phenyl)-benzothiazol-6-ol, (E)-4-(2-(6-(2-(2-(2-[¹⁸F]-fluoroethoxy)ethoxy)pyridin-3-yl)vinyl)-N,N-dimethylbenzenamine([¹⁸F]AV-19), [¹⁸F][4-(2-{4-[2-(2-fluoro-ethoxy)-ethoxy]-phenyl}-vinyl)-phenyl]-methyl-amine, [¹⁸F]{4-[2-(4-{2-[2-(2-fluoro-ethoxy)-ethoxy}-ethoxy]-phenyl)-vinyl]-phenyl}-methyl-amine, [¹⁸F][(4-{2-[4-(2-{2-[2-(2-fluoro-ethoxy)-ethoxy]-ethoxy}-ethoxy)-phenyl]-vinyl}-phenyl)-methyl-amine, and [¹⁸F][[4-(2-{4-[2-(2-{2-[2-(2-fluoro-ethoxy)-ethoxy]-ethoxy}-ethoxy)-ethoxy]-phenyl}-vinyl)-phenyl]-methyl-amine. In one aspect, the ¹⁸F-labelled compound is selected from [¹⁸F]FDG, [¹⁸F]-fluoro-DOPA, [¹⁸F]-fluoroestradiol, 3′-[¹⁸F]-fluorothymidine, 5-[¹⁸F]fluorouracil, [¹⁸F]fluorodopamine, [¹⁸F]fluoronorepinephrine, 2β-carbomethoxy-3β-(4-iodophenyl)nortropane ([¹⁸F]CFT), and N-[¹⁸F]-fluoropropyl-2β-carbomethoxy-3β-(4-iodophenyl)nortropane ([¹⁸F]FP-CIT). In a preferred aspect of the invention, the ¹⁸F-labelled compound is [¹⁸F]FDG.

¹⁸F-labelled compounds which are most at risk of radiolysis are those employed with the minimum amount of non-radioactive carrier compound present, for example where the non-radioactive compound is also biologically active, and is hence expected to compete with the ¹⁸F-labelled compound in vivo. At such no-carrier-added or high specific activity levels, where the radioactive concentration is relatively high, the risk of radiolysis is increased.

By “gentisic acid” is meant 2,5-dihydroxybenzoic acid:

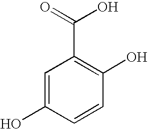

Gentisic acid and salts thereof such as sodium gentisate are commercially available from a wide range of suppliers, for example, Sigma-Aldrich Ltd, UK.

By the term “biocompatible cation” is meant a positively charged counterion which forms a salt with an ionised, negatively charged group, where said positively charged counterion is also non-toxic and hence suitable for administration to the mammalian body, especially the human body. Examples of suitable biocompatible cations include: the alkali metals sodium or potassium; the alkaline earth metals calcium and magnesium; and the ammonium ion. Preferred biocompatible cations are sodium and potassium, most preferably sodium. Preferably, the compositions of the present invention comprise gentisic acid or sodium gentisate, which may be used alone or in admixture.

The term “effective stabilizing amount” means an amount effective to stabilise the ¹⁸F-labelled compound against radiolysis. This means that the gentisic acid or salt thereof is the principal means of stabilization. Other stabilisers could however be present in the composition, but the gentisic acid or salt thereof is the predominant means of stabilisation.

Preferably, the gentisic acid or salt thereof is the sole stabiliser present within the radiopharmaceutical composition. The gentisic acid or salt thereof is suitably used at a concentration of 0.01 to 10.0 mg/ml, preferably 0.1 to 5.0 mg/ml, most preferably 0.5 to 5.0 mg/ml, with 2.5 mg/ml being especially preferred. Since increasing concentrations of gentisic acid will tend to lower the pH of the composition, adjustment of the pH or use of a buffer may be necessary at higher gentisic acid concentrations.

The “aqueous biocompatible carrier medium” is a fluid, especially a liquid, in which the ¹⁸F-labelled compound is suspended or dissolved, such that the composition is physiologically tolerable, i.e. can be administered to the mammalian body without toxicity or undue discomfort. The aqueous biocompatible carrier medium is suitably an injectable carrier liquid such as sterile, pyrogen-free water for injection; an aqueous solution such as saline (which may advantageously be balanced so that the final product for injection is either isotonic or not hypotonic); an aqueous solution of one or more tonicity-adjusting substances (e.g. salts of plasma cations with biocompatible counterions), sugars (e.g. glucose or sucrose), sugar alcohols (e.g. sorbitol or mannitol), glycols (e.g. glycerol), or other non-ionic polyol materials (e.g. polyethyleneglycols, propylene glycols and the like. For the radiopharmaceutical compositions of the present invention, the pH of the composition is suitably controlled by use of an appropriate aqueous biocompatible carrier medium to be suitable for intravenous injection, suitably in the range 4.0 to 9.5, more suitably 4.5 to 8.5, preferably 4.5 to 7.0, most preferably 4.5 to 6.3.

When the radiopharmaceutical is [¹⁸F]FDG, the aqueous biocompatible carrier medium is preferably a mixed aqueous solvent solution of up to 5% (v/v) ethanol with the remaining percentage being an aqueous buffer solution as required by the European Pharmacopeia, such as phosphate buffer.

The radioactive concentration (RAC) of the ¹⁸F in the medium is in the range 10 to 100,000 MBq/ml. Preferably the RAC is in the range 10 to 25,000 MBq/ml. The higher the RAC, the greater the risk of radiolysis, and hence the greater the importance of the effective stabilisers of the present invention. In normal practice the RAC at the time of production is the highest, with radioactive decay meaning that the RAC is considerably lower by the time that formulation, testing, packaging and distribution to the customer have taken place.

The radiopharmaceutical compositions of the present invention are suitably supplied in a clinical grade syringe or a container which is provided with a seal which is suitable for single or multiple puncturing with a hypodermic needle (e.g. a crimped-on septum seal closure) whilst maintaining sterile integrity. Such containers may contain single doses (a “unit dose”) or multiple patient doses.

Suitable containers comprise a sealed vessel which permits maintenance of sterile integrity and/or radioactive safety, whilst permitting addition and withdrawal of solutions by syringe. A preferred such container is a septum-sealed vial, wherein the gas-tight closure is crimped on with an overseal (typically of aluminium). Such containers have the additional advantage that the closure can withstand vacuum if desired e.g. to change the headspace gas or degas solutions.

When the radiopharmaceutical is supplied in a multiple dose container, preferred such containers comprise a single bulk vial (e.g. of 10 to 30 cm³ volume) which contains enough radiopharmaceutical for multiple patient doses. Unit patient doses can thus be withdrawn into clinical grade syringes at various time intervals during the viable lifetime of the bulk vial preparation to suit the clinical situation.

Radiopharmaceutical syringes designed to contain a single human dose, or “unit dose” and are therefore preferably a disposable or other syringe suitable for clinical use. Such syringes may optionally be provided with a syringe shield to protect the operator from radioactive dose. Suitable such radiopharmaceutical syringe shields are known in the art, and various designs are commercially available, and preferably comprise either lead or tungsten.

The radiopharmaceutical composition may optionally further comprise additional components such as an antimicrobial preservative, pH-adjusting agent or filler. By the term “antimicrobial preservative” is meant an agent which inhibits the growth of potentially harmful micro-organisms such as bacteria, yeasts or moulds. The antimicrobial preservative may also exhibit some bactericidal properties, depending on the dose. The main role of the antimicrobial preservative(s) of the present invention is to inhibit the growth of any such micro-organism in the radiopharmaceutical composition. Suitable antimicrobial preservative(s) include: the parabens, i.e. methyl, ethyl, propyl or butyl paraben or mixtures thereof; benzyl alcohol; phenol; cresol; cetrimide and thiomersal. Preferred antimicrobial preservative(s) are the parabens.

The term “pH-adjusting agent” means a compound or mixture of compounds useful to ensure that the pH of the radiopharmaceutical composition is within acceptable limits (approximately pH 4.0 to 8.5) for human or mammalian administration. Suitable such pH-adjusting agents include pharmaceutically acceptable buffers, such as tricine, phosphate buffer or TRIS [i.e. tris(hydroxymethyl)aminomethane], and pharmaceutically acceptable bases such as sodium carbonate, sodium bicarbonate or mixtures thereof. For [¹⁸F] FDG, a preferred buffer is phosphate buffer.

By the term “filler” is meant a pharmaceutically acceptable bulking agent which may facilitate material handling during product production. Suitable fillers include inorganic salts such as sodium chloride, and water soluble sugars or sugar alcohols such as sucrose, maltose, mannitol or trehalose.

The radiopharmaceutical compositions of the present invention may be prepared under aseptic manufacture conditions to give the desired sterile, pyrogen-free product. The radiopharmaceutical compositions may also be prepared under non-sterile conditions, followed by terminal sterilisation using e.g. gamma-irradiation; autoclaving; dry heat; membrane filtration (sometimes called sterile filtration); or chemical treatment (e.g. with ethylene oxide). The ¹⁸F-labelled compound is suitably prepared from a precursor. The “precursor” suitably comprises a non-radioactive analogue of the synthetic compound having an element within its chemical structure (Y) which is designed so that chemical reaction with a convenient chemical form of the ¹⁸F radioisotope occurs at Y, and can be conducted in the minimum number of steps (ideally a single step), and without the need for significant purification (ideally no further purification) to give the desired radioactive product. Such precursors are can conveniently be obtained in good chemical purity. Suitable precursors and their preparation are well known in the art and are reviewed, for example, in Handbook of Radiopharmaceuticals, Radiochemistry and Applications, Ed M. J. Welch and C. S. Redvanly, Pub. John Wiley and Sons Ltd, UK.

The source of the ¹⁸F is most preferably the [¹⁸F]fluoride ion, but in some cases an electrophilic source of ¹⁸F may be used such as [¹⁸F]fluorine or [¹⁸F]-CH₃COOF, or [¹⁸F]—OF₂. [¹⁸F]FDG is routinely manufactured using chemistry based on that described in Hamacher et al, Journal of Nuclear Medicine, 27, (1986), pages 235-283. However, the method of manufacturing the ¹⁸F-labelled compound is not considered to be part of the present invention.

A stabilised radiopharmaceutical composition according to the invention, is preferably stored in an environment from which oxygen gas has been removed.

By the phrase “environment from which oxygen gas has been removed” is meant that appropriate steps have been taken to keep the level of oxygen to the absolute minimum:
(a) when the radiopharmaceutical composition is in solution, oxygen gas has been displaced from the solution and steps are taken to ensure that the headspace gas over the solution is maintained oxygen-free. That is because the environment encompasses both the solution itself and the gas atmosphere that the solution comes into contact with;
(b) when the radiopharmaceutical composition is being prepared, oxygen-free solutions and reaction vessels are employed.

The oxygen gas removal can be achieved by various methods known in the art, for example. prolonged purging of the biocompatible carrier solution with a chemically unreactive gas so that any dissolved oxygen is displaced; freeze-thaw degassing of the biocompatible carrier solution with a chemically unreactive gas or lyophilisation where the atmosphere employed is such an unreactive gas.

By the term “chemically unreactive gas” is meant a gas which would be used in chemistry to provide an “inert atmosphere” as is known in the art. Such a gas does not undergo facile oxidation or reduction reactions (e.g. as would oxygen and hydrogen respectively), or other chemical reactions with organic compounds (as would e.g. chlorine), and is hence compatible with a wide range of synthetic compounds without reacting with the synthetic compound, even on prolonged storage over many hours or even weeks in contact with the gas. Suitable such gases include nitrogen or the inert gases such as helium or argon. Preferably the chemically unreactive gas is nitrogen or argon. Most preferably, the chemically unreactive gas is heavier than air, which maintains a blanket over the stabiliser composition. Hence, a preferred chemically unreactive gas is argon. In order to ensure that ingress of oxygen gas into the de-oxygenated solution does not occur, the headspace gas over the stabiliser is either maintained under a positive pressure of the unreactive gas, or the stabiliser is kept in a gas-tight container (as described above), with the headspace gas being a chemically unreactive gas. Pharmaceutical grade chemically unreactive gases are commercially available.

In a further aspect, the present invention provides a method for preparation of a stabilised radiopharmaceutical composition, which comprises mixing:
(i) an ¹⁸F-labelled compound in a biocompatible carrier medium; with
(ii) an effective stabilizing amount of gentisic acid or a salt thereof with a biocompatible cation;
   wherein the radioactive concentration of the ¹⁸F in the carrier medium is in the range 10 to 100,000 MBq/ml and the pH of the resultant composition is in the range 4.0 to 9.5.

The timing of the introduction of the gentisic acid or salt thereof should be such that the mixing takes place as soon as possible after the production of the ¹⁸F-labelled compound, since the longer the ¹⁸F-labelled compound is in solution in the absence of a stabiliser, the greater the risk of radiolysis.

It is preferred that the gentisic acid or salt thereof is provided in solution and in an environment from which oxygen gas has been excluded. Methods for the exclusion of oxygen gas are described above. The ¹⁸F-labelled compound in a biocompatible carrier medium and the radiopharmaceutical product may optionally also be maintained in an environment from which oxygen gas has been excluded.

In a further aspect, the present invention provides a method for preparation of a stabilised radiopharmaceutical composition as described above comprising the further step of sterilization. The sterilization step may be effected by subjecting the stabilised radiopharmaceutical composition to a thermal sterilization cycle, or by gamma-irradiation; autoclaving; dry heat; membrane filtration (sometimes called sterile filtration); or chemical treatment (e.g. with ethylene oxide).

In a further aspect, the present invention provides the use of gentisic acid or a salt thereof with a biocompatible cation to stabilise against radiolysis a radiopharmaceutical composition comprising an ¹⁸F-labelled compound in an aqueous biocompatible carrier medium as defined above, wherein the radioactive concentration of the ¹⁸F in the carrier medium is in the range 10 to 100,000 MBq/ml and the pH of the resultant composition is in the range 4.0 to 9.5.

This use is particularly valuable for aqueous biocompatible carrier medium which are in a form suitable for human administration as a radiopharmaceutical, i.e. are in sterile form as described above.

The invention will now be illustrated by way of Example.

### EXAMPLES

Radiochemical purity of [¹⁸F]FDG composition samples was determined at end of synthesis (EOS) and at Expiry i.e. after 10 hours storage at 22° C.±3° C. to determine stability of the composition.

### Methods

### Buffer:

Phosphate buffer, isotonic, pH 5.7.

### [¹⁸F]FDG Composition Synthesis

[¹⁸F]FDG was manufactured on an automated synthesis apparatus (TRACERIab Fx, GE Healthcare, Germany), to form a batch solution of [¹⁸F]FDG in isotonic phosphate buffer (pH 5.7). 2-[¹⁸F]Fluoro-2-deoxy-D-mannose ([¹⁸F]FDM) is a by-product of this synthesis.

To a series of vials was added an amount of stabilizer dissolved in Buffer. The batch solution of [¹⁸F]FDG was then dispensed into these vials and thermally sterilized at 134° C. for 210 seconds.

### Stability Testing

### Thin-Layer Chromatography (TLC) Method

At EOS (within 2 hours), a 100-fold dilution of the test solution was prepared by addition of a 10 μl sample to a 990 μl volume of water for injection. After mixing, a 2 μl sample was applied to a TLC strip.

At 10 hours after synthesis, a 10-fold dilution was made by addition of a 20 μl sample to a 180 μl volume of water for injection. After mixing, a 3 μl sample was applied to a TLC strip.

Within 1 to 3 hours after manufacture, radiochemical purity (RCP) was determined by TLC. The vials were stored at 22° C.±3° C. and at 10 hours after synthesis, RCP was again determined by TLC.

Initially, RCP was also measured by High Performance Liquid Chromatography (HPLC), by injecting a 20 μl sample on a Dionex Carbopac column using 0.1 M NaOH as eluent.

In the additional examples (3 to 6) only TLC was used to determine RCP.

### Results

### Example 1

The [¹⁸F]FDG batch solution had batchsize at EOS of 45 GBq in 19 ml (a RAC of 2370 MBq/ml).

| | | | | | RCP (at EOS + | |
|---|---|---|---|---|---|---|
| | | | RCP (EOS) | | 1 h) | |
| | | Calculated | [¹⁸F]-FDG + [¹⁸F]- | | [¹⁸F]-FDG + | |
| | Volume | amount of | FDM | | [¹⁸F]-FDM | |
| Vial | (ml) | Additive | HPLC | TLC | HPLC | TLC |
| Control | 1.2 | no | 94.8 | 94.3 | 95.5 | 93.2 |
| 3 | 2.0 | 2.5 mg/ml | 97.4 | 96.0 | 97.6 | 95.2 |
| | | Acetone | | | | |
| 4 | 2.0 | 5 mg/ml | 97.4 | 96.5 | 97.8 | 95.8 |
| | | Acetone | | | | |
| 5 | 2.0 | 20 mg/ml | 97.9 | 96.8 | 98.4 | 96.3 |
| | | Acetone | | | | |
| 6 | 2.0 | 2.5 mg/ml | 98.7 | Not | 99.0 | 97.5 |
| | | Ethanol | | determined | | |
| 7 | 2.0 | 5 mg/ml | 98.6 | 98.0 | 99.2 | 97.8 |
| | | Ethanol | | | | |
| 8 | 2.0 | 20 mg/ml | 98.8 | 98.1 | 99.2 | 97.4 |
| | | Ethanol | | | | |
| 9 | 2.0 | 3 mg/ml | 98.3 | 97.4 | 98.5 | 97.0 |
| | | gentisic acid | | | | |
| 10 | 2.0 | 6 mg/ml | 98.7 | 97.5 | 98.8 | 97.5 |
| | | gentisic acid | | | | |

*) Residual solvents: 39 ug/ml EtOH en 32 ug/ml Acetone.

Conclusion: Acetone has a slight stabilising effect. Ethanol and gentisic acid have a better stabilising effect than acetone.

Examples 2, 3 and 4 explored the stabilizing effect of different concentrations of Gentisic Acid (GA) using batches <50 GBq at EOS.

### Example 2

The [¹⁸F]FDG batch solution had batchsize at EOS of 36 GBq in 10.9 ml (a RAC of 3300 MBq/ml).

| | | | RCP | | RCP | |
|---|---|---|---|---|---|---|
| | | | (EOS) | | (at Expiry) | |
| | | | [¹⁸F]-FDG + | | [¹⁸F]-FDG + | |
| | Volume | | [¹⁸F]-FDM | | [¹⁸F]-FDM | |
| Vial | (ml) | Additive | HPLC | TLC | HPLC | TLC |
| Control | 1.2 | No *) | 98.1 | 96.6 | 95.9 | 94.3 |
| 9 | 1.0 | 0.1 mg/ml GA | 98.1 | 97.1 | 98.5 | 96.5 |
| 11 | 1.0 | 0.25 mg/ml GA | 98.2 | 98.3 | 98.5 | 97.0 |
| 10 | 1.0 | 0.5 mg/ml GA | 98.2 | 97.0 | — | 96.8 |

*) Residual solvents: 122 ug/ml EtOH and 66 ug/ml Acetone.

### Example 3

The [¹⁸F]FDG batch solution had batchsize at EOS of 43 GBq in 17 ml (a RAC of 2500 MBq/ml

| | | | RCP (EOS) | RCP (at Expiry) |
|---|---|---|---|---|
| | | | [¹⁸F]-FDG + | [¹⁸F]-FDG + |
| | | | [¹⁸F]- | [¹⁸F]- |
| | Volume | | FDM | FDM |
| Vial *) | (ml) | Additive | TLC | TLC |
| Control | 1.2 | no *) | 97.5 | 95.7 |
| 10 | 1.0 | 0.05% gentisic | — | 98.0 |
| | | acid | | |
| 11 | 1.0 | 0.1% gentisic acid | 98.7 | 98.4 |

*) 154 μg/ml EtOH + 89 μg/ml Acetone

### Example 4

The [¹⁸F]FDG batch solution had batchsize at EOS of 40 GBq in 17 ml (a RAC of 2350 MBq/ml).

| | | | RCP (EOS) | RCP (at Expiry) |
|---|---|---|---|---|
| | | | [¹⁸F]-FDG + | [¹⁸F]-FDG + |
| | Volume | | [¹⁸F]-FDM | [¹⁸F]-FDM |
| Vial *) | (ml) | Additive | TLC | TLC |
| QC | | no *) | 94.4 | 93.7 |
| 4 | | 1 mg/ml gentisic | 97.8 | 97.8 |
| | | acid | | |
| 5 | | 2 mg/ml gentisic | 97.8 | 97.9 |
| | | acid | | |

39 μg/ml EtOH + 32 μg/ml Acetone

### Conclusion

In three small batches (total activity <50 GBq at EOS) the effect of increasing amounts of gentisic acid was explored by comparing successively the ranges 0.1-0.5 mg/ml, 0.5-1 mg/ml and 1-2 mg/ml of gentisic acid. In all cases the higher amount was found to be more effective.

Examples 5 and 6 were performed to determine the stabilizing effect of Gentisic Acid in larger batch sizes and higher activity concentrations.

### Example 5

The [¹⁸F]FDG batch solution had batchsize at EOS of 43.3 GBq in 12.1 ml (a RAC of 3578 MBq/ml).

| | | | | | | RCP |
|---|---|---|---|---|---|---|
| | | | | | | (EOS + |
| | | | | | | 10 h) |
| | | | | | RCP | [¹⁸F]- |
| | | | | | (EOS) | FDG + |
| | Vol. | | Total | | [¹⁸F]-FDG + | [¹⁸F]- |
| | FDG | | vol. | Stabilizers in | [¹⁸F]-FDM | FDM |
| Vial | (ml) | Addition | (mL) | final mixture | TLC | TLC |
| 1 | 1.2 | 100 ul final | 1.3 | no additive *) | 95.5 | 94.5 |
| | | buffer | | | | |
| 4 | 1.2 | 120 ul of | 1.3 | 1 mg/ml EtOH | 97.4 | 97.2 |
| | | 2.5% EtOH | | | | |
| 5 | 1.2 | 120 ul of 5% | 1.3 | 1.5 mg/ml EtOH | 97.8 | 97.5 |
| | | EtOH | | | | |
| 6 | 1.2 | 120 ul 27 mg/ml | 1.3 | 2.5 mg/ml GA *) | 98.4 | 98.5 |
| | | GA | | | | |

*) residual ethanol from synthesis approx. 0.1 mg/ml

### Conclusion

The absolute amount of stabilizer in vial 5 and 6 is almost the same (approximately 2 mg/ml). The RCP at E0S+10 h is 1% higher for GA, which indicates that GA is a better stabiliser than ethanol.

### Example 6

The [¹⁸F]FDG batch solution had batchsize at EOS of 85.6 GBq in 15.8 ml (a RAC of 5418 MBq/ml).

| | | | | | | RCP |
|---|---|---|---|---|---|---|
| | | | | | RCP | (EOS + |
| | | | | | (EOS) | 10 h) |
| | Vol. | | Total | | [¹⁸F]-FDG + | [¹⁸F]-FDG + |
| | FDG | | vol. | Stabilizers in final | [¹⁸F]-FDM | [¹⁸F]-FDM |
| Vial | (ml) | Addition | (mL) | mixture | TLC | TLC |
| 1 | 2.0 | 200 ul | 2.2 | 0.5 mg/ml EtOH | 95.7 | 94.2 |
| | | buffer | | (no additive) | | |
| 6 | 2.0 | 200 ul 27 mg/ml | 2.2 | 2.5 mg/ml GA *) | 98.3 | 98.1 |
| | | GA | | | | |

*) vial #6 contains also residual ethanol from synthesis (approx. 0.5 mg/ml)

1. A stabilised radiopharmaceutical composition which comprises:
(i) an ¹⁸F-labelled compound;
(ii) an effective stabilizing amount of gentisic acid or a salt thereof with a biocompatible cation;
(iii) an aqueous biocompatible carrier medium;
wherein the radioactive concentration of the ¹⁸F in the carrier medium is in the range 10 to 100,000 MBq/ml and the pH of the composition is in the range 4.0 to 9.5.

2. A radiopharmaceutical composition according to claim 1 wherein the ¹⁸F-labelled compound is selected from [¹⁸F]FDG, [¹⁸F]-fluoro-DOPA, [¹⁸F]-fluoroestradiol, 3′-[¹⁸F]-fluorothymidine, 5-[¹⁸F]fluorouracil, [¹⁸F]fluorodopamine, [¹⁸F]fluoronorepinephrine, 2β-carbomethoxy-3β-(4-iodophenyl)nortropane ([¹⁸F]CFT), N—[¹⁸F]-fluoropropyl-2β-carbomethoxy-3β-(4-iodophenyl)nortropane ([¹⁸F]FP-CIT)2-(1-(6-((2-[¹⁸F]fluoroethyl)(methyl)amino)naphthalen-2-yl)ethylidene)malonitrile ([¹⁸F]FDDNP), 2-(3-[¹⁸F]-fluoro-4-methylamino-phenyl)-benzothiazol-6-ol, 2-(2-[¹⁸F]-fluoro-4-methylamino-phenyl)-benzothiazol-6-ol, (E)-4-(2-(6-(2-(2-(2-[¹⁸F]fluoroethoxy)ethoxy)pyridin-3-yl)vinyl)-N,N-dimethylbenzenamine([¹⁸F]AV-19), [¹⁸F][4-(2-{4-[2-(2-fluoro-ethoxy)-ethoxy]-phenyl}-vinyl)-phenyl]-methyl-amine, [¹⁸F]{4-[2-(4-{2-[2-(2-fluoro-ethoxy)-ethoxy]-ethoxy}-phenyl)-vinyl]-phenyl}-methyl-amine, [¹⁸F][(4-{2-[4-(2-{2-[2-(2-fluoro-ethoxy)-ethoxy]-ethoxy}-ethoxy)-phenyl]-vinyl}-phenyl)-methyl-amine, and [¹⁸F][[4-(2-{4-[2-(2-{2-[2-(2-fluoro-ethoxy)-ethoxy]-ethoxy}-ethoxy)-ethoxy]-phenyl}-vinyl)-phenyl]-methyl-amine.

3. A radiopharmaceutical composition according to claim 2 wherein the ¹⁸F-labelled compound is [¹⁸F]FDG.

4. A radiopharmaceutical composition according to claim 1 wherein the amount of gentisic acid is 0.01 to 10.0 mg/ml.

5. A radiopharmaceutical composition according to claim 1 wherein the pH of the composition is in the range 4.5 to 8.5.

6. A radiopharmaceutical composition according to claim 1 wherein the radioactive concentration of the ¹⁸F in the carrier medium is in the range 10 to 25,000 MBq/ml.

7. A method for preparation of a stabilised radiopharmaceutical composition, which comprises mixing:
(i) an ¹⁸F-labelled compound in a biocompatible carrier medium; with
(ii) an effective stabilizing amount of gentisic acid or a salt thereof with a biocompatible cation;
wherein the radioactive concentration of the ¹⁸F in the carrier medium is in the range 10 to 100,000 MBq/ml and the pH of the resultant composition is in the range 4.0 to 9.5.

8. A method according to claim 7 comprising the further step of sterilization.

9. A method according to claim 7 wherein the ¹⁸F-labelled compound is selected from [¹⁸F]FDG, [¹⁸F]-fluoro-DOPA, [¹⁸F]-fluoroestradiol, 3′-[¹⁸F]-fluorothymidine, 5-[¹⁸F]fluorouracil, [¹⁸F]fluorodopamine, [¹⁸F]fluoronorepinephrine, 2β-carbomethoxy-3β-(4-iodophenyl)nortropane ([¹⁸F]CFT), N-[¹⁸F]fluoropropyl-2β-carbomethoxy-3β-(4-iodophenyl)nortropane ([¹⁸F]FP-CIT)2-(1-(6-((2-[¹⁸F]fluoroethyl)(methyl)amino)naphthalen-2-yl)ethylidene)malonitrile ([¹⁸F]FDDNP), 2-(3-[¹⁸F]-fluoro-4-methylamino-phenyl)-benzothiazol-6-ol, 2-(2-[¹⁸F]-fluoro-4-methylamino-phenyl)-benzothiazol-6-ol, (E)-4-(2-(6-(2-(2-(2-[¹⁸F]fluoroethoxy)ethoxy)pyridin-3-yl)vinyl)-N,N-dimethylbenzenamine([¹⁸F]AV-19), [¹⁸F][4-(2-{4-[2-(2-fluoro-ethoxy)-ethoxy]-phenyl}-vinyl)-phenyl]-methyl-amine, [¹⁸F]{4-[2-(4-{2-[2-(2-fluoro-ethoxy)-ethoxy]-ethoxy}-phenyl)-vinyl]-phenyl}-methyl-amine, [¹⁸F][(4-{2-[4-(2-{2-[2-(2-fluoro-ethoxy)-ethoxy]-ethoxy}-ethoxy)-phenyl]-vinyl}-phenyl)-methyl-amine, and [¹⁸F][[4-(2-{4-[2-(2-{2-[2-(2-fluoro-ethoxy)-ethoxy]-ethoxy}-ethoxy)-ethoxy]-phenyl}-vinyl)-phenyl]-methyl-amine.

10. A method according to claim 9 wherein the ¹⁸F-labelled compound is [¹⁸F]FDG.

11. A method according to claim 7 wherein the amount of gentisic acid is 0.01 to 10.0 mg/ml.

12. A method according to claim 7 wherein the pH of the composition is in the range 4.5 to 8.5.

13. A method according to claim 7 wherein the radioactive concentration of the ¹⁸F in the carrier medium is in the range 10 to 25,000 MBq/ml.

14. (canceled)
